(19) United States
(12) Patent Application Publication
(10) Pub. No.: US 2015/0174020 A1
(43) Pub. Date: Jun. 25, 2015

(54) COMPOSITIONS CONTAINING TITANATE SILICONE NETWORKS

(71) Applicant: Avon Products, Inc., Suffern, NY (US)

(72) Inventors: Allwyn Colaco, Morristown, NJ (US); Mohannad Abdo, Clifton, NJ (US)

(73) Assignee: Avon Products, Suffern, NY (US)

(21) Appl. No.: 14/411,147

(22) PCT Filed: Mar. 13, 2014
(86) PCT No.: PCT/US2014/026308

§ 371 Date: Dec. 24, 2014

Related U.S. Application Data
(60) Provisional application No. 61/793,154, filed on Mar. 15, 2013.

(51) Int. Cl. A61K 8/31; A61Q 1/00; A61K 8/898; A61K 8/58; A61Q 1/06

(52) U.S. Cl. CPC A61K 8/31; A61K 8/58; A61Q 1/06; A61K 8/898; A61Q 1/00; A61K 2800/48

(57) ABSTRACT

The invention relates generally to gels or viscous fluids formed by the coordination of certain amino-functionalized siloxanes with titanates and the use of the same in cosmetic compositions, including for providing long-wearing, comfortable films on a keratinous surface (e.g., lips). The gels or viscous fluids may exhibit unique rheological properties, including shear thickening (dilatancy) and/or rheopecty, at low shear rates which may lead to self-leveling effects, including renewable shine; and may exhibit high gloss and low shear.

### RELATED APPLICATIONS

This application claims priority benefit to U.S. Provisional Patent Application Ser. No. 61/793,154, filed on Mar. 15, 2013, the entirety of which is herein incorporated by reference for all purposes.

### FIELD OF INVENTION

The invention relates generally to gels and viscous fluids formed by the coordination of certain amino-functionalized siloxanes with titanates and the use of the same in cosmetic compositions, including for providing long-wearing, comfortable films on a keratinous surface (e.g., lips). The gels and viscous fluids formed may exhibit unique rheological properties, including shear thickening (dilatancy) and/or rheopecty, at low shear rates which may lead to self-leveling effects, including renewable shine.

### BACKGROUND OF THE INVENTION

It has long been considered desirable to provide cosmetic products, such as lipsticks, which impart colored films to the skin. Conventionally, color cosmetics comprise pigments dispersed in a base of fats, oils, or waxes. However, a notable disadvantage of traditional color cosmetic products is the propensity to transfer from the lips on contact with napkins, drinking glasses, clothing and the like.

Various polymeric film formers have been added to color cosmetics to improve the substantivity to the skin and enhance transfer resistance. Improvements in transfer resistance have been achieved using various silicone resins, including MQ resins. Japanese Patent Application 61-161211 and Japanese Patent Application 61-158913, both assigned to Shiseido, discloses cosmetic compositions comprising MQ resins having improved resistance to sweat and oil and which do not transfer to objects such as drinking glasses.

There is, however, room for improvement. There is an ongoing need for cosmetic compositions that are long-wearing and transfer resistant. It is therefore an object of the present invention to provide cosmetic compositions, such as lipstick, lip gloss, mascara, and nail enamels, which have reduced propensity to transfer on contact with surfaces such as napkins, clothes, utensils, and drinking glasses.

The foregoing discussion is presented solely to provide a better understanding of nature of the problems confronting the art and should not be construed in any way as an admission as to prior art nor should the citation of any reference herein be construed as an admission that such reference constitutes “prior art” to the instant application.

### SUMMARY OF THE INVENTION

In accordance with the foregoing objectives and others, the present invention provides methods and compositions for forming cosmetic films on a human integument, including human hair and skin, and in particular the lips. The cosmetic films may be long-wearing and/or transfer resistant and/or comfortable (e.g., low tack) and/or provide high, renewable shine.

In one aspect of the invention, a gel or viscous fluid composition is provided. The gel or viscous fluid is formed from titanium alkoxide (e.g., about 3-15% by weight), a hydroxyl-functionalized amodimethicone polymer (e.g., about 5-25% by weight), and an oil (e.g., about 50-95% by weight) which gels with the titanium alkoxide and hydroxyl-functionalized amodimethicone polymer. In one embodiment, a film formed by the gel or viscous fluid is characterized by a gloss value above about 55 gloss units, preferably above about 60 gloss units, and most preferably above about 65 gloss units, as measured at 20°. In another embodiment, a film formed by the gel or viscous fluid is characterized by a tack force below about 200 g, typically below about 150 g, and ideally below about 100 g. In some embodiments, the inventive compositions are in the form of lip products (e.g., a lip gloss or lip stick) that is characterized as having high shine (e.g., about 60-90 gloss units) and low tack (e.g., about 50-125 g).

The titanium alkoxide may, for example, have the form Ti(OR₁)₃(OR₂) where R₁ is independently selected at each occurrence from C₈₋₃₄ (e.g., C₁₀₋₂₈ or C₁₂₋₂₆ or C₁₄₋₂₄ or C₁₆₋₂₂) hydrocarbons (e.g., branched, straight chained, or cyclic alkyl, alkenyl, etc.), and R₂ is selected from C₁₋₇ hydrocarbons or C₂₋₄ hydrocarbons (e.g., branched, straight chained, or cyclic alkyl, alkenyl, alkynyl, aryl, etc.), including without limitation, methyl, ethyl, propyl, isopropyl, butyl, iso-butyl, tert-butyl, pentyl, hexyl, heptyl, phenyl, and benzyl. R₁ may, for example, be a C₁₀₋₂₈ or C₁₂₋₂₆ or C₁₄₋₂₄ or C₁₆₋₂₂ straight chained or branched alkyl or alkenyl at one, two, or all three occurrences, including, without limitation, groups such as lauryl, tridecyl, isotridecyl, myristyl, pentadecyl, cetyl, isocetyl, palmitoleyl, heptadecyl, stearyl, isostearyl, oleyl, nonadecyl, arachidyl, heneicosyl, behenyl, erucyl, lignoceryl, and ceryl, to name a few. In one implementation, R₁ is isostearyl at each occurrence and/or R₂ is isopropyl.

The hydroxyl-functionalized amodimethicone polymer ideally comprises end-capped hydroxyl groups. It may be characterized by a functional group equivalent weight (FGEW) of amine of about 12-19 milli-equivalents per gram. The polymer may further be characterized by a viscosity between about 500 and about 2,000 cps (or about 900 and about 1,300 cps) at 25° C. The hydroxyl-functionalized amodimethicone polymer may, for example, have the structure:

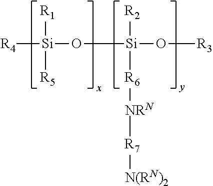

where R₁, R₂, and R₅ are independently selected at each occurrence selected from C₁₋₈ (or C₁₋₆ or C₁₋₄) hydrocarbon radicals (e.g., alkyl, alkenyl, aryl, benzyl, etc.), a group L₁, or a group R₈; R₆ and R₇ are independently divalent C₁₋₆ (or C₁₋₄ or C₁₋₂) hydrocarbon radicals (e.g., alkyl), R₃ is hydrogen, a group L₁, or a group R₈; and R₄ is —OH, —OR*, or a group L₁ where L₁ is a radical of the form —X^{a}—(CR*₂)ₖ—(CR*═CR*)ₗ—(CR*₂CR*₂O)ₘ—X^{b}—(CR*₂CR*₂O)ₙ—(CR*₂)ₚ—(CR*═CR*)ₚ—OH, where X^{a} and X^{b} are independently either a bond (i.e., absent), —O—, —S—, —NH—, —NR^{N}—; and where “k,” “l,” “m,” “n,” “p,” and “q” are independently at each occurrence integers from 0-4 (e.g., one, two, three, or four). R₈ has the structure:

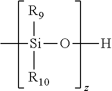

where R₉ and R₁₀ are independently selected from C₁₋₈ (or C₁₋₆ or C₁₋₄) hydrocarbon radicals (e.g., alkyl, alkenyl, aryl, benzyl, etc.), and where “x,” “y,” and “z” are integers from 0-500, or in the case of “x,” “y,” integers from 1-500 (or 2-300 or 3-250), selected such that the polymer has a functional group equivalent weight (FGEW) of amine of about 12-19 or 12-17 milli-equivalents per gram and a viscosity between about 500 and about 2,000 cps at 25° C.,

where R* and R^{N} are independently selected at each occurrence from hydrogen or C₁₋₈ (or C₁₋₆ or C₁₋₄) hydrocarbon radicals (e.g., alkyl, alkenyl, aryl, benzyl, etc.). The polymer comprises at least two groups —OH.

In some implementations, R₁, R₂, and R₅ are methyl, and/or R₃ is hydrogen, and/or R₄ is —OH, and/or R₆ and R₇ are independently groups selected from —(CH₂)ₖ—, where k is an integer from 1-6 (or from 1-4 or from 1-2).

The gel or viscous fluid may further comprise an amine-functionalized siloxane polymer (e.g., from about 0.05% to about 2.5% by weight) of the form:

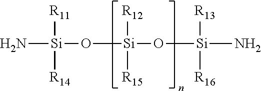

where R₁₁-R₁₆ are independently selected at each occurrence from C₁₋₈ (or C₁₋₆ or C₁₋₄ or C₁₋₂) hydrocarbon radicals (e.g., alkyl, alkenyl, aryl, benzyl, etc.), for example methyl, or where R₁₁-R₁₆ may independently be a group R₈, where R₈ has the structure:

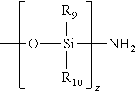

where R₉ and R₁₀ are independently selected from C₁₋₈ (or C₁₋₆ or C₁₋₄ or C₁₋₂) hydrocarbon radicals (e.g., alkyl, alkenyl, aryl, benzyl, etc.), for example methyl, and where “n” and “z” are integers from 0-300, and in the case of “n,” from 1-300, selected such that the polymer has a viscosity between about 10 cps and about 2,500 cps (or between 50 and 1,500 cps or between 100 and 1,000 cps) at 25° C. Such a polymer has been found useful to fine tune the rheology of the gel or viscous fluid or hardness of the gel, for example, to improve the spreadability or pay off of the product.

The oil may comprise for example, silicone oils, hydrocarbon oils, fatty alcohols, ester oils, and/or optionally hydrogenated polyolefins, including polyolefins formed from the polymerization of monomers of the form (R^{a})(R^{b})C═C(R^{c})(R^{d}) where R^{a}, R^{b}, R^{c}, R^{d} are independently selected from hydrogen and C₁₋₆ (or C₁₋₄ or C₁₋₂) hydrocarbon radicals, including without limitation polyisobutylene, hydrogenated polyisobutylene, polybutene, and/or hydrogenated polybutene.

The gel or viscous fluid may be in the form of cosmetic composition. The cosmetic composition may comprise conventional cosmetic ingredients and auxiliaries. For example, the cosmetic composition may comprise pigments (e.g., from about 1% to about 35% by weight). The cosmetic composition may further comprise film forming polymers, waxes, co-gellants, thickeners, stabilizers, emulsifiers, humectants, emollients, organic sunscreens, pH adjusters, antioxidants, active ingredients, preservatives, and combinations thereof (e.g., collectively or individually present in an amount from about 0.01% to about 35% by weight).

A cosmetic composition is provided comprising a gel or viscous fluid formed from isopropyl triisostearyl titanate (e.g., about 3-15% by weight), a hydroxyl-functionalized amodimethicone polymer (e.g., about 5-25% by weight), an oil (e.g., about 50-95% by weight) which gels with the isopropyl triisostearyl titanate and hydroxyl-functionalized amodimethicone polymer, and optionally an end-capped amine-functionalized siloxane polymer (e.g., from 0.05% to about 2.5% or to about 5% or to about 10% by weight); wherein the hydroxyl-functionalized amodimethicone polymer may, for example, have the structure:

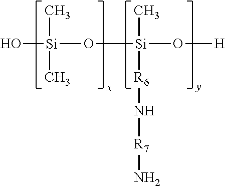

wherein R₆ and R₇ are independently groups-(CH₂)ₖ—, where k is an integer from 1-6 or 1-4 or 1-3 or 1-2; and where “x,” “y” are integers from 1-500 (or 2-350 or 3-250), selected such that the polymer has a functional group equivalent weight (FGEW) of amine of about 12-19 or about 12-17 milli-equivalents per gram and a viscosity between about 500 and about 2,500 cps (or about 500 and about 1,800 cps) at 25° C.; and wherein the end-capped amine-functionalized siloxane polymer may, for example, have the form:

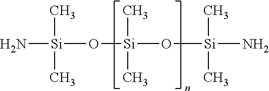

where “n” is an integer from 1-300 (or from 1-150 or from 2-100 or from 3-50), selected such that the polymer has a viscosity between about 10 cps and about 2,500 cps (or between 50 and 1,500 cps or between 100 and 1,000 cps) at 25° C. In one embodiment, “n” is selected to provide a FGEW of the end-capped amine-functionalized siloxane polymer between about 100 to about 3,000 g/mol, more particularly, from about 130 to 2,200 g/mol, or from about 200 to about 750 g/mol, or from about 300 to about 500 g/mol.

In one embodiment, a film formed by the composition is characterized by a gloss value above about 55 gloss units, preferably above about 60 gloss units, and most preferably above about 65 gloss units, as measured at 20°. In another embodiment, a film formed by the composition is characterized by a tack force below about 200 g, preferably below about 150 g, and most preferably below about 100 g.

The gel or viscous fluid and cosmetic compositions according to the invention display unique rheology which may improve the slip and feel, comfort, and/or payoff, and which may provide for renewable shine and self-leveling effects. The rheology may be characterized by shear thickening (dilatant) behavior at a first range of shear rates (e.g., from about 1 s⁻¹ to about 15 s⁻¹ or from about 1 s⁻¹ to about 20 s⁻¹ from about 1 s⁻¹ to about 25 s⁻¹) and a shear thinning rheology at a second range of shear rates greater than said first range of shear rates (e.g., from about 35 s⁻¹ to about 100 s⁻¹ or from about 40 s⁻¹ to about 100 s⁻¹ from about 50 s⁻¹ to about 100 s⁻¹), as shown in FIG. 1. In other embodiments, the cosmetic composition or gels may be characterized by a rheotectic rheology at a first shear rate and a thixotropic rheology at a second shear rate greater than said first shear rate. For example, the cosmetic composition or gels may be characterized by a rheotectic rheology at a first shear rate of 10 s⁻¹ and a thixotropic rheology at a second shear rate of 100 s⁻¹, as shown in FIG. 2. However, the invention is not intended to be limited to any particular rheology, unless otherwise indicated.

The gelled compositions of the invention are preferably stable over at least one (or at least two) freeze-thaw cycles from −4° F. to 110° F. or from −4° F. to 150° F. In one embodiment, the gelled compositions of the invention are stable 110° F. for two weeks. In one embodiment, the gelled compositions of the invention are stable 150° F. for two weeks.

In another aspect of the invention, a method is provided for forming a film on a human integument (e.g., hair, lashes, nails, skin, lips, etc.) comprising applying to said integument a deposit of a composition according to the invention to form a film thereon. The film may, for example, be self-leveling upon the removal of a shear force to regenerate a glossy film. By way of example, the gloss of a lipcolor may be renewed, at least partially, by rubbing the lips together to create shear, and upon cessation of the rubbing the film may begin to flow and even out imperfections in its surface. The smoothing of surface imperfections will enhance the gloss (e.g., as measured visually or by Bossa Nova). This property may also aid in long-wear properties as the substantivity of the film to the skin may be renewed as the film is caused to flow and re-adhere to the skin.

These and other aspects of the present invention will become apparent to those skilled in the art after a reading of the following detailed description of the invention, including the appended claims.

### BRIEF DESCRIPTION OF THE FIGURES

FIG. 1 shows the shear rate dependent rheology of an exemplary cosmetic composition according to the invention. As illustrated, the composition exhibits shear thickening (dilatant) behavior at low shear rates (e.g., less than about 30 s⁻¹) and shear thinning behavior at higher shear rates (e.g., greater than about 35 s⁻¹).

FIG. 2 shows the time dependent rheology of an exemplary cosmetic composition according to the invention. As illustrated, the composition exhibits thixotropy at low shear rates (e.g., 10 s⁻¹) and exhibits unusual rheopecty at higher shear rates (e.g., 100 s⁻¹).

FIG. 3 shows the results of Gloss Test measurements of a composition according to the invention compared to three commercially available “high shine” products.

FIG. 4 shows the results of Tack Test measurements (force (g)) of a composition according to the invention compared to three commercially available “high shine” products.

### DETAILED DESCRIPTION

All amounts provided in terms of weight percentage are relative to the entire composition unless otherwise stated. Unless otherwise provided, the term “alkyl” is intended to embrace straight-chained, branched, or cyclic hydrocarbons, particularly those having from one to 20 carbon atoms, and more particularly C₁₋₁₈ or C₁₋₁₆ or C₁₋₁₂ or C₁₋₁₀ or C₁₋₆ hydrocarbons. The term “lower alkyl” includes, without limitation methyl, ethyl, propyl, isopropyl, butyl, n-butyl, iso-butyl, tert-butyl, pentyl, neo-pentyl, amyl, and hexyl, including cyclized analogues thereof. Unless otherwise provided, the term “alkenyl” is intended to embrace straight-chained, branched, or cyclic hydrocarbons, particularly those having from one to 20 carbon atoms, and more particularly C₁₋₁₈ or C₁₋₁₆ or C₁₋₁₂ or C₁₋₁₀ or C₁₋₆ hydrocarbons, which may include one, two, three, or more unsaturated carbon-carbon bonds, which may be in the E or Z configuration.

The compositions of the invention are useful for application to the human integumentary system, including, skin, lips, nails, hair, and other keratinous surfaces. As used herein, the term “keratinous surface” refers to keratin-containing portions of the human integumentary system, which includes, but is not limited to, skin, lips, hair (including eyebrows and eyelashes), and nails (toenails, fingernails, cuticles, etc.) of mammalians, preferably humans. A “keratin fiber” includes hair of the scalp, eyelashes, eyebrows, facial hair, and body hair such as hair of the arms, legs, etc.

The compositions according to the invention are in the form of gel or viscous fluid. By “gel” is meant a material that is semi-solid. By “fluid” is meant a material that flows. By “viscous fluid” is meant a fluid having a viscosity from about 2,500 to about 750,000 cps. The gel or viscous fluid may be incorporated into cosmetic compositions or may themselves constitute a cosmetic composition. The following description, including the ingredients and amounts specified, is equally applicable to gels or viscous fluids unless otherwise noted.

The gel or viscous fluid is formed from (1) titanium alkoxide, (2) a hydroxyl-functionalized amodimethicone polymer, and (3) an oil which gels with the titanium alkoxide and hydroxyl-functionalized amodimethicone polymer. The titanium alkoxide may constitute about 3-15% (or about 5-10%) by weight of the composition, a hydroxyl-functionalized amodimethicone polymer may constitute about 5-25% (or about 8-15%) by weight of the composition, and an oil which gels with the titanium alkoxide and hydroxyl-functionalized amodimethicone polymer may constitute about 50-95% by weight of the composition, depending on the desired hardness and elasticity.

Any titanium alkoxide capable of forming a gelling network with the amodimethicone polymer is suitable. The titanium alkoxide may, for example, be a mixed alkoxide, including those of the form Ti(OR₁)₃(OR₂), where R₁ is independently selected at each occurrence from C₈₋₃₄ (e.g., C₁₀₋₂₈ or C₁₂₋₂₆ or C₁₄₋₂₄ or C₁₆₋₂₂) hydrocarbons (e.g., branched, straight chained, or cyclic alkyl, alkenyl, etc.), and R₂ is selected from C₁₋₇ hydrocarbons or C₂₋₄ hydrocarbons (e.g., branched, straight chained, or cyclic alkyl, alkenyl, alkynyl, aryl, etc.), including without limitation, methyl, ethyl, propyl, isopropyl, butyl, iso-butyl, tert-butyl, pentyl, hexyl, heptyl, phenyl, and benzyl. R₁ may, for example, be a C₁₀₋₂₈ or C₁₂₋₂₆ or C₁₄₋₂₄ or C₁₆₋₂₂ straight chained or branched alkyl or alkenyl at one, two, or all three occurrences, including, without limitation, groups such as lauryl, tridecyl, isotridecyl, myristyl, pentadecyl, cetyl, isocetyl, palmitoleyl, heptadecyl, stearyl, isostearyl, oleyl, nonadecyl, arachidyl, heneicosyl, behenyl, erucyl, lignoceryl, and ceryl, to name a few. In one embodiment R₁ is branched C₁₂₋₂₂ hydrocarbon. In one embodiment, R₁ is isostearyl at each occurrence and/or R₂ is selected is isopropyl.

In one embodiment, the titanate is isopropyl-triisostearyl titanate (CAS: 61417-49-0). While isopropyl-triisostearyl titanate, sometimes referred to as ITT, is often used to functionalize the surface of pigments (e.g., metal oxides), it will be understood that in the present invention the isopropyl-triisostearyl titanate is not associated with (i.e., bound to) the surface of a pigment. Rather it is believed, without wishing to be bound by any particular theory, that the isopropyl-triisostearyl titanate forms a coordinate bonding network, having long-range order, with the amodimethicone component. The amino groups are thought to form a coordinate bond through their lone pair with the titanium atom. The isopropoxy groups may form bridges between adjacent titanium atom groups. This theory forms no part of the invention unless otherwise stated.

The hydroxyl-functionalized amodimethicone polymer ideally comprises end-capped hydroxyl groups. This is in contrast to other amodimethicones that are end-capped with, for example, alkyl (e.g., methyl) groups. Some amondimethicones are end-capped with alkoxy (e.g., methoxy or ethoxy) groups and are also considered suitable in the practice of the invention because these functionalities are hydrolyzed in the presence of water to generate hydroxyl groups. The hydroxyl-functionalized amodimethicone polymer may be characterized by a functional group equivalent weight (FGEW) of amine of about 12-19 milli-equivalents per gram. The polymer may further be characterized by a viscosity between about 500 and about 2,000 cps (or about 900 and about 1,300 cps) at 25° C. One suitable material is Shin Etsu's KF-874 hydroxy-end capped amodimethicone which is believed to have a viscosity of about 650 cps and a FGEW of amine of about 19 milli-equivalents per gram. Another suitable material is Mirasil ADM-211 which is believed to have a viscosity of about 900-1300 cps and a FGEW of amine of about 12-17 milli-equivalents per gram.

The hydroxyl-functionalized amodimethicone polymer may, for example, have the structure:

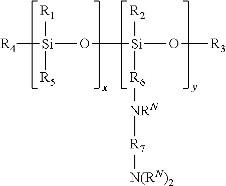

where R₁, R₂, and R₅ are independently selected at each occurrence selected from C₁₋₈ (or C₁₋₆ or C₁₋₄) hydrocarbon radicals (e.g., alkyl, alkenyl, aryl, benzyl, etc.), a group L₁, or a group R₈; R₆ and R₇ are independently divalent C₁₋₆ (or C₁₋₄ or C₁₋₂) hydrocarbon radicals (e.g., alkyl), R₃ is hydrogen, a group L₁, or a group R₈; and R₄ is —OH, —OR*, or a group L₁ where L₁ is a radical of the form —X^{a}—(CR*₂)ₖ—(CR*═CR*)ₗ—(CR*₂CR*₂O)ₘ—X^{b}—(CR*₂CR*₂O)ₙ—(CR*₂)ₚ—(CR*═CR*)_{q}—OH, where X^{a} and X^{b} are independently either a bond (i.e., absent), —O—, —S—, —NH—, —NR^{N}—; and where “k,” “l,” “m,” “n,” “p,” and “q” are independently at each occurrence integers from 0-4 (e.g., one, two, three, or four). R₈ has the structure:

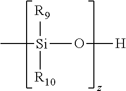

where R₉ and R₁₀ are independently selected from C₁₋₈ (or C₁₋₆ or C₁₋₄) hydrocarbon radicals (e.g., alkyl, alkenyl, aryl, benzyl, etc.), and where “x,” “y,” and “z” are integers from 0-500, or in the case of “x,” “y,” integers from 1-500 (or 2-300 or 3-250), selected such that the polymer has a functional group equivalent weight (FGEW) of amine of about 12-19 or 12-17 milli-equivalents per gram and a viscosity between about 500 and about 2,000 cps at 25° C.,

where R* and R^{N} are independently selected at each occurrence from hydrogen or C₁₋₈ (or C₁₋₆ or C₁₋₄) hydrocarbon radicals (e.g., alkyl, alkenyl, aryl, benzyl, etc.). R* and R^{N} may, for example, independently be hydrogen, methyl, ethyl, propyl, butyl, or phenyl. In some embodiments, the polymer comprises at least two groups —OH. In some embodiment, the polymer comprises more than two groups —OH.

In some implementations, R₁, R₂, and/or R₅ are methyl, and/or R₃ is hydrogen, and/or R₄ is —OH, and/or R₆ and R₇ are independently groups selected from —(CH₂)ₖ—, where “k” is an integer from 1-6 (or from 1-4 or from 1-2), including embodiments where “k” is one, two, or three at each occurrence.

The gel or viscous fluid may further comprise an amine-functionalized siloxane polymer (e.g., from about 0.05% to about 2.5% or to about 5% or to about 10% by weight or from about 0.1% to about 1% by weight) of the form:

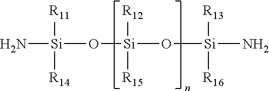

where R₁₁-R₁₆ are independently selected at each occurrence from C₁₋₈ (or C₁₋₆ or C₁₋₄ or C₁₋₂) hydrocarbon radicals (e.g., alkyl, alkenyl, aryl, benzyl, etc.), for example methyl, or where R₁₁-R₁₆ may independently be a group R₈, where R₈ has the structure:

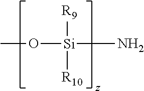

where R₉ and R₁₀ are independently selected from C₁₋₈ (or C₁₋₆ or C₁₋₄ or C₁₋₂) hydrocarbon radicals (e.g., alkyl, alkenyl, aryl, benzyl, etc.), for example methyl, and where “n” and “z” are integers from 0-300, and in the case of “n,” from 1-300, selected such that the polymer has a viscosity between about 1 cps and about 2,500 cps (or between 50 and 1,500 cps or between 100 and 1,000 cps) at 25° C. Such a polymer has been found useful to fine tune the rheology or hardness of the gel. When a group R₈ is not present, the additional compound is a linear end-capped amine-functionalized siloxane polymer. One example is Shin Etsu's KF8010. It has been observed that the addition of minor components such as preservatives (e.g., at levels of about 0.001% to about 1% by weight) may weaken or collapse the gel, in which case an amount of such an amino end-capped siloxane can restore the original elasticity or consistency of the gel. In some embodiment, the preservative is caprylyl glycol which has been found to be particularly disruptive to the gel and, at 0.5% can collapse a stable gel. When caprylyl glycol (or other protic or polar ingredients) is employed, often the amount of titanate tetramer or the amount of amodimethicone polymer will be used at increased levels sufficient to restore the consistency of the gel or fluid to that which it would otherwise be in the absence of the caprylyl glycol (or other protic or polar ingredients).

In other embodiments, the amine-functionalized silicone polymer may comprise primary amine groups on alkyl chains which form side chains or terminal residues on a dimethicone or siloxane polymer. An example is aminopropyl dimethicone.

The oil may comprise for example, silicone oils, hydrocarbon oils (e.g., mineral oil), fatty alcohols, ester oils, and/or optionally hydrogenated polyolefins. Suitable polyolefins may be formed from the homo- or co-polymerization of monomers of the form (R^{a})(R^{b})C═C(R^{c})(R^{d}) where R^{a}, R^{b}, R^{c}, R^{d} are independent selected from hydrogen and C₁₋₆ (or C₁₋₄ or C₁₋₂) hydrocarbon radicals, including without limitation, methyl groups, ethyl group, propyl groups, and phenyl groups. Examples of such polyolefin oils include, without limitation polyisobutylene, hydrogenated polyisobutylene, polybutene, and/or hydrogenated polybutene. The amount of oil is not particularly limited and may be selected based on the desired consistency of the gel. In some embodiment, the oil may comprise from 25% to about 99% by weight of the composition. More typically, the oil will comprise from about 50-95% by weight or from about 60% to about 90% or from about 65% to about 85% or from about 70% to about 80% by weight of the composition.

In some embodiments a wax may be added to the composition, including as part of the oily component to be gelled in the titanium/amondimethicone network. Suitable waxes include, without limitation, linear polyethylene, microcrystalline petroleum wax, carnauba wax, lignite wax, ouricouri wax, rice bran wax, castor wax, mortar wax, stearone, acrawax, bayberry wax, castor wax, Japan wax, ozokerite, beeswax, candelilla wax, petrolatum, ceresin wax, cocoa butter, illipe butter, esparto wax, shellac wax, ethylene glycol diesters or triesters of C₁₈-C₃₆ fatty acids, cetyl palmitate, paraffin wax, hard tallow, lanolin, lanolin alcohol, cetyl alcohol, glyceryl monostearate, sugarcane wax, jojoba wax, stearyl alcohol, silicone waxes, and combinations thereof. In some embodiment, the composition will include from about 0.1% to about 15% by weight wax. In some embodiment, the composition will include less than 5% by weight wax. In some embodiment, the composition will include less than 1% by weight wax. In some embodiment, the composition will free of wax. In one embodiment, the composition is in the form of a wax-free, gel-based mascara.

In one embodiment, a cosmetic composition comprises a gel or viscous formed from isopropyl triisostearyl titanate (e.g., about 3-15% by weight), a hydroxyl-functionalized amodimethicone polymer (e.g., about 5-25% by weight), an oil (e.g., about 50-95% by weight) which gels with the isopropyl triisostearyl titanate and hydroxyl-functionalized amodimethicone polymer, and at least one additional ingredient selected from pigments, fillers, polymeric film-formers, thickeners, co-gellants, stabilizers, emulsifiers, pH adjusters, antioxidants, and preservatives. In this embodiment, the composition optionally comprises an end-capped amine-functionalized siloxane polymer (e.g., in an amount from 0.05% to about 2.5% or to about 5% or to about 10% by weight) to restore or adjust the firmness and/or consistency and/or rheology of the gel. The hydroxyl-functionalized amodimethicone polymer may, for example, have the structure:

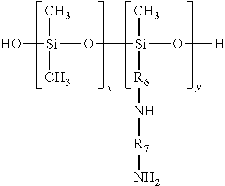

wherein R₆ and R₇ are independently groups-(CH₂)ₖ—, where “k” is an integer from 1-6 or 1-4 or 1-3 or 1-2; and where “x,” “y” are integers from 1-500 (or 2-350 or 3-250), selected such that the polymer has a functional group equivalent weight (FGEW) of amine of about 12-19 or about 12-17 milli-equivalents per gram and a viscosity between about 500 and about 2,500 cps (or about 500 and about 1,800 cps) at 25° C. The end-capped amine-functionalized siloxane polymer may, for example, be a linear siloxane of the form:

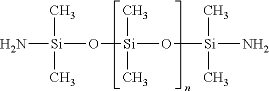

where “n” is an integer from 1-300 (or from 1-150 or from 2-100 or from 3-50), selected such that the polymer has a viscosity between about 1 cps and about 2,500 cps (or between 5 and about 2,000 or between about 50 and 1,500 cps or between 100 and 1,000 cps) at 25° C.

The gel or viscous liquid may be in the form of a cosmetic composition. The cosmetic composition may comprise conventional cosmetic ingredients and auxiliaries. For example, the cosmetic composition may comprise pigments (e.g., from about 1% to about 35% by weight). The cosmetic composition may further comprise film forming polymers, waxes, co-gellants, thickeners, stabilizers, emulsifiers, humectants, emollients, organic sunscreens, pH adjusters, antioxidants, active ingredients, preservatives, and combinations thereof (e.g., collectively or individually present in an amount from about 0.01% to about 35% by weight).

The cosmetic composition may have a viscosity from about 100 cps up to about 2,000,000 cps, as measured at a shear rate of 10 s⁻¹ at 25° C. Typically, the cosmetic composition will have a viscosity from about 1,000 cps up to about 1,000,000 cps, as measured at a shear rate of 10 s⁻¹ at 25° C. More typically, the cosmetic composition will have a viscosity from about 2,000 cps up to about 500,000 cps, as measured at a shear rate of 10 s⁻¹ at 25° C.

In some embodiments, the composition may further include a hydroxyl-functionalized silicone polymer (e.g., a dimethiconol) which may have the structure:

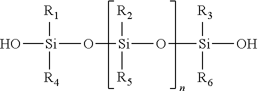

where R₁-R₆ is independently a C₁₋₁₀ or C₁₋₆ or C₁₋₄ hydrocarbon (aliphatic or aromatic) substituent. R₁-R₆ may be selected from methyl, ethyl, propyl, isopropyl, butyl, isobutyl, tert-butyl, pentyl, hexyl, vinyl, allyl, ethylenyl, propenyl, hexenyl, phenyl, benzyl, cyclopentyl, and cyclohexyl, as well as halogenated (fluoro, chloro, etc.) and perhalogenated (e.g., perfluorinated) analogs thereof, and wherein each of R₁-R₆ may further include 1-4 (e.g., one, two, three, or four) substituents selected from hydroxyl, alkoxyl, polyalkyleneoxide (e.g., polyethylene oxide), amino, alkyl amino, and dialkylamino Integer “n” may range from 2 up to 200, 500, 800, 1,000, 1,500, 2,000 or more, such that the viscosity is, for example, from 20 to 1,000,000 cps or greater (or from 50 to 500,000 cps or from 100 to 5,000 cps) at 25° C. In one embodiment at least one of R₂ and R₅ is methyl. In one embodiment R₂ and R₅ are both methyl. In one embodiment R₁-R₆ are all methyl. In one embodiment, the silicone polymer is dimethiconol.

In some embodiments, the composition may further include an MQ resin, where “M” refers to units of the form R₃SiO_{1/2} and “Q” refers to units of the form SiO_{4/2}. The MQ resins of the invention may further have some content (typically minor, or up to 5 mole %) of “D” units of the form R₂SiO and “T” units of the form RSiO_{3/2}, as well as other silicon-containing units of the form RₙSiXₘO_{y}, where R is a C₁₋₁₀ or C₁₋₆ or C₁₋₄ hydrocarbon (aliphatic or aromatic) substituent (such as methyl, ethyl, propyl, butyl, pentyl, hexyl, vinyl, allyl, ethylenyl, propenyl, hexenyl, phenyl, cyclopentyl, cyclohexyl, etc.), and X is a functional group H, OH, Cl or OR. In some embodiments, R is methyl. In some embodiments, R is methyl at 90% or more of the occurrences. In some embodiments, R is methyl at 95% or more of the occurrences. The ratio of M to Q units may be, for example, about 5:1 to about 1:5 or about 2:1 to about 1:2 or about 3:2 to about 2:3. MQ resins may have the INCI name Trimethylsiloxysilicate. The silanol content of the MQ resin may range from about 0.1% to about 10% by weight, or from about 0.2% to about 5% by weight, or from about 1% to about 3% by weight of the MQ resin. The hydroxyl group may be in the form of a dimethylhydroxysiloxy unit of the form (HO)(CH₃)₂SiO_{1/2}. One suitable MQ resin is BELSIL® TMS 803 from Wacker which is the co-hydrolysis product of tetraalkoxysilane (Q unit) and trimethylethoxysilane (M unit). The chemical structure of this MQ resin can be seen as a three-dimensional network of polysilic acid units, which are endblocked with trimethylsilyl groups and retain some residual ethoxy and hydroxy functional groups.

In another embodiment, the composition may further include a fluorinated MQ resin. US Patent Pub. 2006/0229424, the disclosure of which is hereby incorporated by reference, describes reacting MQ resins with fluoroalkylsilanes. In another embodiment, the MQ resin may be a MQ/Propyl silsesquioxane (T propyl) resin, as disclosed in, for example, U.S. Pat. No. 7,803,358, WO2005075542 and W0200507556, the disclosures of which are hereby incorporated by reference. In another embodiment, composition may comprise a Trimethylsiloxysilicate/Dimethiconol Crosspolymer (NCI). Such material may comprise a crosspolymer of an MQ resin with Dimethiconol. This polymer provides additional hydroxyl groups at the end-caps. One example is Dow Corning's DC 7-4405.

The cosmetic compositions of the invention may comprise any conventional components, including pigments and colorants, fillers and cosmetic powders, additional film formers, gelling agents, thickeners, conditioners, actives, solvents, emulsifiers, humectants, emollients, pH adjusters, antioxidants, preservatives, fragrances, and the like.

For example, the compositions may, for example, comprise (1) from about 1% to about 75% (e.g., about 1-10%, about 10-20%, about 30-40%, about 40-50%, or about 50-60%) by weight of an additional oil, including without limitation hydrocarbons (e.g., isododecane), fatty alcohols, ester oils, and silicone oils; (2) from about 0.5% to about 20% (e.g., about 0.5-2.5%, or about 2.5%-5%, or about 5-10%, or about 10-15%, or about 15-20%) by weight of a film-forming polymer; (3) from about 1% to about 25% (e.g., about 1%-5%, or about 5-10%, or about 10-15%, or about 15-20%) by weight of a gelling agent, such as Isododecane (and) Disteardimonium (and) Hectorite (NCI); (4) from about 0.1% to about 15% (e.g., about 0.1-1%, or about 1-2.5%, or about 2.5-5%, or about 5-10%, or about 10-15%) by weight of a wax, such as beeswax, carnauba, ozokerite, or hydrogenated lanolin; (5) from about 1% to about 35% (e.g., about 8% to about 20%) by weight pigments; and/or (6) from about 0.01% to about 2.5% (e.g., about 0.1-0.5%, about 0.5-1%, or about 1-2.5%) by weight of a preservative, such as a paraben (e.g., methyl paraben and propylparaben) or caprylyl glycol.

The composition may also comprise colorants such as dyes, pigments and lakes. As used herein, the term “pigments” embraces lakes and fillers such as talc, calcium carbonate, etc. Exemplary inorganic pigments include, but are not limited to, inorganic oxides and hydroxides such as magnesium oxide, magnesium hydroxide, calcium oxide, calcium hydroxides, aluminum oxide, aluminum hydroxide, iron oxides (α-Fe₂O₃, γ-Fe₂O₃, Fe₃O₄, FeO) and iron hydroxides including red iron oxide, yellow iron oxide and black iron oxide, titanium dioxide, titanium lower oxides, zirconium oxides, chromium oxides, chromium hydroxides, manganese oxides, manganese hydroxides, cobalt oxides, cobalt hydroxides, cerium oxides, cerium hydroxides, nickel oxides, nickel hydroxides, zinc oxides and zinc hydroxides and composite oxides and composite hydroxides such as iron titanate, cobalt titanate and cobalt aluminate and the like. Preferably, the inorganic oxide particles may be selected from silica, alumina, zinc oxide, iron oxide and titanium dioxide particles, and mixtures thereof. In one embodiment, the pigments have a particle size from 5 nm to 150 microns, or from 5 nm to 50 microns, or from 10 nm to 25 microns. In some embodiments, the particle size (median) will be less than about 5 microns or less than 1 micron.

Additional exemplary color additive lakes include, for example: D&C Red No. 19 (e.g., CI 45170, CI 73360 or CI 45430); D&C Red No. 9 (CI 15585); D&C Red No. 21 (CI 45380); D&C Orange No. 4 (CI 15510); D&C Orange No. 5 (CI 45370); D&C Red No. 27 (CI 45410); D&C Red No. 13 (CI 15630); D&C Red No. 7 (CI 15850:1); D&C Red No. 6 (CI 15850:2); D&C Yellow No. 5 (CI 19140); D&C Red No. 36 (CI 12085); D&C Orange No. 10 (CI 45475); D&C Yellow No. 19 (CI 15985); FD&C Red #40 (CI #16035); FD&C Blue #1 (CI #42090); FD&C Yellow #5 (CI #19140); or any combinations thereof.

Suitable fillers may include talc, silica, alumina, zinc stearate, mica, kaolin, nylon (in particular orgasol) powder, polyethylene powder, polypropylene powder, acrylates powders, Teflon, starch, boron nitride, copolymer microspheres such as Expancel (Nobel Industrie), Polytrap (Dow Corning), and silicone resin microbeads (Tospearl from Toshiba).

Other fillers that may be used in the compositions of the invention include inorganic powders such as chalk, fumed silica, fumed alumina, calcium oxide, calcium carbonate, magnesium oxide, magnesium carbonate, Fuller's earth, attapulgite, bentonite, muscovite, phlogopite, synthetic mica, lepidolite, hectorite, biotite, lithia mica, vermiculite, aluminum silicate, aluminum magnesium silicate, diatomaceous earth, starch, alkyl and/or trialkyl aryl ammonium smectites, chemically modified magnesium aluminum silicate, organically modified montmorillonite clay, hydrated aluminum silicate, hydrated silica, fumed aluminum starch octenyl succinate barium silicate, calcium silicate, magnesium silicate, strontium silicate, metal tungstate, magnesium, silica alumina, zeolite, barium sulfate, calcined calcium sulfate (calcined gypsum), calcium phosphate, fluorine apatite, hydroxyapatite, ceramic powder, metallic soap (zinc stearate, magnesium stearate, zinc myristate, calcium palmitate, and aluminum stearate), colloidal silicon dioxide; organic powder, cyclodextrin, methyl polymethacrylate powder, copolymer powder of styrene and acrylic acid, benzoguanamine resin powder, and poly(ethylene tetrafluoride) powder.

The compositions may include natural or synthetic film-forming polymers. Suitable polymeric film formers include polyolefins, silicone polymers (e.g., dimethicones, dimethiconols, amodimethicones, silicone resins, etc.), (meth)acrylates, alkyl(meth)acrylates, polyurethanes, fluoropolymers, silicone polyurethanes, and silicone acrylates such as acrylates/dimethicone copolymers. In some embodiments, it may be desirable to add a hydrophilic or water-soluble film former (e.g., cellulosics, polysaccharides, polyquaterniums (such as polyquaternium-37 (INCI), etc.) to the composition to improve spreading, emulsion stability, aesthetic look and feel, etc. another polymer is VP/Eicosene Copolymer. Elastomers formed from ethylene, propylene, butylene, and/or styrene monomers may also be useful.

The compositions of the invention may include a cosmetically or dermatologically acceptable vehicle, which may be in the form of, for example, a serum, a cream, a lotion, a gel, or a stick, and may comprise an emulsion (e.g., water-in-oil, oil-in-water, water-in-silicone, silicone-in-water, polyol-in-silicone, silicone-in-polyol emulsion, etc.), or may comprise an aqueous or ethanolic vehicle, silicone (e.g., cyclomethicone, dimethicone, etc.), hydrocarbon (e.g., petrolatum, isododecane, etc.), ester oil (isopropyl myristate, myristyl myristate, or the like. The vehicle may be anhydrous and may comprise oils, such as dimethicones, hydrocarbons (e.g., isododecane), petrolatum, ester oils, and the like. The vehicle may further comprise an emulsifier, gelling agent, structuring agent, rheology modifier (e.g., a thickener), film former, or the like. The vehicle may comprise from about 5% to about 99% by weight of the composition.

The compositions may further include an emulsifier. The amount of emulsifier will typically be from about 0.001 to about 10% by weight, but preferably will range from about 0.01 to about 5% by weight, and most preferably about 0.1 to about 1% by weight, based upon the total weight of the composition. The emulsifier may be ionic, zwitterionic, or nonionic. Suitable emulsifiers include those of the polyethoxylated type (e.g., polyoxyethylene ethers or esters), polydiorganosiloxane-polyoxyalkylene block copolymers (e.g., dimethicone copolyol), Steareth-20, Steareth-21, fatty alcohols (e.g., Cetearyl Alcohol), Polyoxethylene sorbitan fatty acid esters (i.e., polysorbates), and Hydrogenated Castor Oil, to name a few. Additional emulsifiers are provided in the NCI Ingredient Dictionary and Handbook 11th Edition 2006, the disclosure of which is hereby incorporated by reference.

The composition may further include an additional gelling agent. The gelling agent may comprise, for example, a silicone resin, including Dimethicone/Vinyl Dimethicone crosspolymer, silicone T-resins, ETPEA, polyamides, cellulose ethers (e.g., methyl cellulose or ethyl cellulose) and the like. Thickeners such as Acrylates Copolymer, hydroxyalkyl cellulose (e.g., hydroethylcellulose), carboxymethylcellulose, carbomers, and vegetable gums such as xanthan gum and gum Arabic may be included. A stabilizer such as dibutyl lauroyl glutamide may also be included, typically in an amount from about 0.1% to about 10% of the composition.

The cosmetic compositions of the invention may optionally include one or more agents that provide or enhance shine. Shine enhancing agents will typically have a refractive index greater than about 1.4, preferably greater than about 1.5 when measured as a film at 25° C. Suitable shine enhancing agents include without limitation, polyols, fatty esters, silicone phenylpropyldimethylsiloxysilicate, polybutene, polyisobutene, hydrogenated polyisobutene, hydrogenated polycyclopentadiene, propyl phenyl silsesquioxane resins; lauryl methicone copolyol, perfluorononyl dimethicone, dimethicone/trisiloxane, methyl trimethicone, and combinations thereof. In one embodiment, the composition will comprise a shine-enhancing agent in an amount from about 0.1% to about 10% by weight, based on the total weight of the composition.

In another embodiment, the compositions of the present invention may also include one or more of the following: a skin penetration enhancer (e.g., squalene); an emollient, such as isopropyl myristate, petrolatum; volatile or non-volatile silicones oils (e.g., methicone, dimethicone, cyclomethicone pentamer), ester oils, mineral oils, and fatty acid esters; a humectant, such as glycerin, hexylene glycol or caprylyl glycol; a skin plumper, such as palmitoyl oligopeptide, collagen, collagen and/or glycosaminoglycan (GAG) enhancing agents; an exfoliating agent; and an antioxidant (e.g., TDPA).

The composition may comprise humectants such as polyols (e.g., glycols), including without limitation, glycerin, propylene glycol, ethoxydiglycol, butylene glycol, pentylene glycol, hexylene glycol, caprylyl glycol, and the like. These will typically be added in amount from about 0.001 to about 5% by weight. When a humectant is present, it may or may not be necessary to add an amount of a linear amine end-capped siloxane to achieve the desired consistency of the gel.

The compositions of the invention may optionally include additional skin benefit agents such as emollients (dimethicone oils, ester oils, or hydrocarbon oils), humectants (e.g., polyols, including propylene glycol, glycerin, etc.), antioxidants (e.g., BHT, ascorbic acid, sodium ascorbate, ascorbyl palmitate, beta-carotene, etc.), vitamins (e.g., tocopherol, tocopheryl acetate, etc.), alpha-hydroxy acids (e.g., glycolic acid), beta-hydroxy acids (e.g., salicylic acid), retinoids (e.g., retinoic acid, all-trans-retinoic acid, retinaldehyde, retinol, and retinyl esters such as acetates or palmitates), other anti-aging ingredients (e.g., collagen stimulators), and depigmenting agents (e.g., TDPA).

A sunscreen may be included to protect the skin from damaging ultraviolet rays. In an illustrative embodiment of the present disclosure, the sunscreen provides both UVA and UVB protection, by using either a single sunscreen or a combination of sunscreens. Among the sunscreens that can be employed in the present compositions are avobenzone, cinnamic acid derivatives (such as octylmethoxy cinnamate), octyl salicylate, oxybenzone, octocrylene, titanium dioxide, zinc oxide, or any mixtures thereof. The sunscreen may be present from about 1 wt % to about 30 wt % of the total weight of the composition.

Other suitable components include those agents that provide a prophylactic or therapeutic benefit to skin. Particular mention may be made of alpha-hydroxy acids, beta hydroxyl acids, ascorbic acid or Vitamin C and derivatives thereof (e.g., C₁-C₈ esters thereof); retinoids such as retinol (Vitamin A) and the esters thereof (e.g., C₁-C₈ esters, such as palmitate), retinoic acid and the derivatives thereof, hyaluronic acid, chemical sun screens useful in the cosmetic field including any UVA and UVB filter useful in the cosmetic field including mixtures thereof and blends with physical filters including, but not limited to metal oxide particles such as titanium oxides and/or zinc oxides. Additional benefit agents include botanicals, thiodipropionic acid (TDPA) and esters thereof; (e.g., retinoic acid, all-trans-retinoic acid, retinaldehyde, retinol, and retinyl esters such as acetates or palmitates, and others); alpha-hydroxy acids (e.g., glycolic acid), beta-hydroxy acids (e.g., salicylic acid and salicylates); exfoliating agents (e.g., glycolic acid, 3,6,9-trioxaundecanedioic acid, etc.), depigmenting agents (e.g., hydroquinone, kojic acid, etc.) estrogen synthetase stimulating compounds (e.g., caffeine and derivatives); compounds capable of inhibiting 5 alpha-reductase activity (e.g., linolenic acid, linoleic acid, finasteride, and mixtures thereof); antioxidants (e.g., BHT, ascorbic acid, sodium ascorbate, ascorbyl palmitate, beta-carotene, thiodipropionic acid, vitamin E, etc.), barrier function enhancing agents (e.g., ceramides, glycerides, cholesterol and its esters, alpha-hydroxy and omega-hydroxy fatty acids and esters thereof, etc.); collagenase inhibitors; and elastase inhibitors; to name a few. These benefit agents will typically be present, if at all, in amounts between about 0.001% and about 10% by weight of the composition.

Other additives include: vitamins, such as tocopherol and ascorbic acid; vitamin derivatives such as ascorbyl monopalmitate, tocopheryl acetate, and Vitamin E palmitate; metal chelating agents such as EDTA or salts thereof; and pH adjusters (citric acid, ethanolamine, sodium hydroxide, etc.).

The compositions typically comprises a preservative or anti-microbial agent, for example, methylchloroisothiazolinone, methylisothiazolinone, methylparaben, propylparaben, phenoxyethanol, or caprylyl glycol.

The composition can be applied as often as necessary to impart the desired cosmetic finish, color or appearance to the lip, nails, hair, lashes and/or skin. A composition according to the invention is expected to achieve transfer resistance, comfort, and gloss, and remain on the integument (hair, skin, lips, nails, etc.) for a long-wear period such as from about 1 to about 24 hours. The composition is also expected to maintain transfer resistance for a period such as from about 1 hour to about 24 hours. Typically, a long-wear period and/or transfer resistance period may be from about 2-24 hours, or about 4-24 hours, or 8-24 hours.

A variety of evaluation methods of transfer resistance and long wearing properties are known in the cosmetic arts, such as in dry blot, oil blot, and rub tests. For example, U.S. Pat. No. 6,071,503 discloses various methods of evaluating cosmetic properties, the disclosure of which is hereby incorporated by reference.

Films formed by the gels, viscous fluids, and compositions of the invention provide high shine (e.g., as assessed by a gloss test described in the examples), while at the same time maintaining a comfortable, non-tacky feel on a human integument (e.g., as assessed by a tack test described in the examples). In one embodiment, films formed by the gels, viscous fluids or compositions, are characterized by a gloss value above about 55 gloss units, preferably above about 60 gloss units, and most preferably above about 65 gloss units, as measured at 20°. In another embodiment, films formed by the gels, viscous fluids or compositions, are characterized by a tack force below about 200 g, preferably below about 150 g, and most preferably below about 100 g.

In one embodiment, the composition is intended for use as a non-therapeutic treatment. In another embodiment, the composition is an article intended to be rubbed, poured, sprinkled, or sprayed on, introduced into, or otherwise applied to the human body for cleansing, beautifying, promoting attractiveness, or altering the appearance, in accordance with the US FD&C Act, §201(i).

### Example 1

Gels were constructed from Isopropyl Titanium Trisisostearate (Ken-React® KR® TTS) and the hydroxy end-capped amodimethicone (Mirasil ADM-211) in isododecane oil (IDD). The amount of the titanate and the amodimethicone were varied to study the effect on the rheology of the gel. The different formulas are shown below in Table 1.

TABLE 1

| | Mirasil ADM- | KenReact KR- | IDD | |
|---|---|---|---|---|
| Sample | 211 | TTS (%) | (%) | Note |
| 1 | 10 | 0 | 90 | thin solution |
| 2 | 10 | 1 | 89 | slightly viscous |
| 3 | 10 | 2.5 | 87.5 | viscous fluid |
| 4 | 10 | 5 | 85 | gel |
| 5 | 10 | 7.5 | 82.5 | highly elastic gel |
| 6 | 10 | 10 | 80 | Elastic gel |
| 7 | 0 | 10 | 90 | thin solution |

As shown in Table 1, gels are not formed from the hydroxyl-end-capped amodimethicone (Mirasil ADM-211) alone (Sample 1) or from the Isopropyl Titanium Trisisostearate (Ken-React® KR® TTS) alone (Sample 7). For the combination, gels were formed at amounts of Isopropyl Titanium Trisisostearate above 2.5% by weight, with the maximum elasticity of the gel occurring between about 5% and about 10% by weight, with excellent results obtained at about 7.5% by weight Isopropyl Titanium Trisisostearate and dropping off slightly at higher amounts.

### Example 2

Compositions according to the invention were constructed from the ingredient shown in Table 2. In each case the Isopropyl Titanium Trisisostearate was Ken-React® KR® TTS and the hydroxy (or alkoxy) end-capped amodimethicone was Mirasil ADM-211. The amount of optional ingredient Shin Etsu KF8010, an amine end-capped dimethicone polymer, was varied from 0-1% to study the effect of the viscosity of the composition.

TABLE 2

| Ingredient | A | B | C | D |
|---|---|---|---|---|
| Ken-React ® KR ® TTS | 8 | 8 | 8 | 8 |
| Mirasil ADM-211 | 12 | 12 | 12 | 12 |
| Shin Etsu KF8010 | 0 | 0.3 | 0.6 | 1 |
| Caprylyl Glycol | 0.5 | 0.5 | 0.5 | 0.5 |
| D&C Red 7 | 2.5 | 2.5 | 2.5 | 2.5 |
| Hydrogenated | q.s | q.s | q.s | q.s |
| Polyisobutene | | | | |
| Total | 100 | 100 | 100 | 100 |
| Viscosity (cps) | 858,000 | 348,000 | 33,000 | 2,400 |

As shown above, the viscosity of the composition is extremely sensitive to small changes in the amount of amino-dual-end amodimethicone polymer. As the percent amino-dual-end amodimethicone was increased, the viscosity of the gels (A and B) decreased to viscous fluids (C and D). This property can be used to fine tune the rheology of the gel of fluid and the pay-off of the composition.

To assess the transfer resistance of each formulation of Table 1, 1 mL drawdowns of each formulation was applied as a film on Vitro skin and allowed to dry for one hour. Droplets of water or artificial sebum were equilibrated on each sample for 15 minutes and then excess liquid was removed. Styrofoam discs attached to a 1 kg weight were rotated once on the sample and the amount of colorant remaining on the Vitro skin and the amount transferred to the Styrofoam disc were evaluated visually. It was found that all of the tested compositions achieved excellent oil-transfer resistance, although the addition of Shin Etsu KF8010 measurably improved the oil transfer-resistance from a grading of 4 for Sample A (average of four trials) to a grading of 5 for each of Samples B-D. The scale ranged from 1-5, with 5 being a grade which exceeds the performance of a positive control benchmark, and 1 being a grade which underperforms a negative control benchmark.

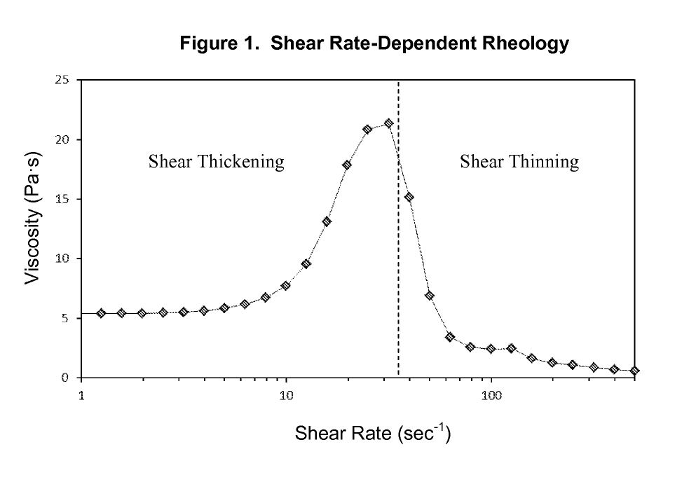

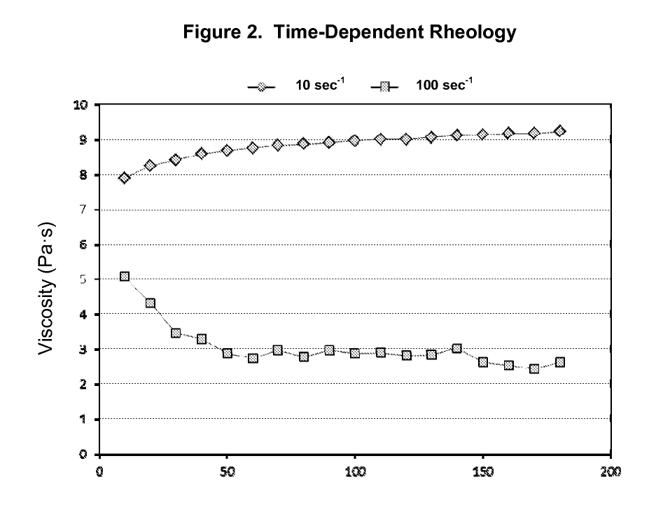

The rheology of sample B was investigated using a standard steel cone rheometer. FIG. 1 illustrates the shear rate dependent viscosity of sample B from shear rates of 0.5 to 500 l/s. As shown in FIG. 1, the composition exhibits unusual rheological behavior in that it is shear thickening (dilatant) up to shear rates of about 30-35 l/s and then it becomes shear thinning as shear rates increase further. Without wishing to be bound by any theory, it is believed that as shear rate reaches a critical point, the coordinate bonds to the titanium metal are broken and the gel rapidly collapses, possibly accounting for the transition to shear thinning behavior. A shown in FIG. 2, the composition exhibits rheopecty at a shear rate of 10 l/s but thioxotropy at a shear rate of 100 l/s. Again, without wishing to be bound by any theory, it is believed that the composition is rheopectic at shear rates normally encountered by rubbing the lips together or other types of slight contact. Under these conditions, the composition is believed to stiffen while under shear and then flow when the force is removed to generate a leveled surface in which imperfections are smoothed out. This self-leveling property provides renewable shine which would be particularly ideal in a lip gloss or lip color cosmetic. It is also believed that the non-Newtonian rheology improves the substantivity on the skin because it allows the interface between the film and the skin to be renewed and minimizes or eliminates flaking of the film.

### Example 3

Gels were prepared according to Table 3 to determine the effect of different oil components. The amounts listed are in % by weight. The compositions were prepared by mixing the KF-867S into each oil and vortexing until uniform. The Ken-React® KR® TTS was then added and vortexed until uniformed and the samples were placed in an oven overnight at 120° F.

TABLE 3

| Ingredient | 8 | 9 | 10 | 11 | 12 | 13 | 14 | 15 | 16 | 17 | 18 |
|---|---|---|---|---|---|---|---|---|---|---|---|
| Ken-React ® KR ® | 5 | 5 | 5 | 5 | 5 | 5 | 5 | 5 | 5 | 5 | 5 |
| TTS | | | | | | | | | | | |
| Shin Etsu KF-867S | 10 | 10 | 10 | 10 | 10 | 10 | 10 | 10 | 10 | 10 | 10 |
| Mineral Oil | 85 | — | — | — | — | — | — | — | — | — | — |
| Jojoba oil | — | 85 | — | — | — | — | — | — | — | — | — |
| Hydrogenated | — | — | 85 | — | — | — | — | — | — | — | — |
| Polyisobutene | | | | | | | | | | | |
| Polybutene | — | — | — | 85 | — | — | — | — | — | — | — |
| Octyldodecyl Stearoyl | — | — | — | — | 85 | — | — | — | — | — | — |
| Stearate | | | | | | | | | | | |
| Diisotearyl fumarate | — | — | — | — | — | 85 | — | — | — | — | — |
| Oleyl | — | — | — | — | — | — | 85 | — | — | — | — |
| Erucate/Tocopherol | | | | | | | | | | | |
| Isononyl/Isononanoate | — | — | — | — | — | — | — | 85 | — | — | — |
| Diisostearyl Malate | — | — | — | — | — | — | — | — | 85 | — | — |
| Triisostearyl | — | — | — | — | — | — | — | — | — | 5 | — |
| Trilinoleate | | | | | | | | | | | |
| Isododecane (IDD) | — | — | — | — | — | — | — | — | — | 85 | — |
| Cyclomethicone | — | — | — | — | — | — | — | — | — | — | 85 |
| pentamer (D5) | | | | | | | | | | | |
| Notes: | ppt | | Ø | Ø | | X | [symbol] | ⊕ | ⊕ | X | ⊕ |

The quality of each gel was visually assessed. In Table 3, the notes are as follows: “ppt” indicates that a solid precipitate was formed; X indicates that no gel was formed; Ø indicates that a thick, sticky opaque gel was formed; \textcircled{×} indicates that a viscous gel was formed but there was discoloration due to reaction; ⊕ indicates that clear to slightly colored viscous gels were formed.

### Example 4

Gels according to the invention were constructed from the ingredient shown in Table 4. In each case the Isopropyl Titanium Trisisostearate was Ken-React® KR® TTS and the hydroxy (or alkoxy) end-capped amodimethicone was Mirasil ADM-211. The amount of optional ingredient Shin Etsu KF8010, an amine end-capped dimethicone polymer, was varied from 0-1% to study the effect of the viscosity of the gel.

TABLE 4

| Ingredient | E | F | G | H | I | J |
|---|---|---|---|---|---|---|
| Ken-React ® KR ® TTS | 8 | 8 | 8 | 8 | 8 | 8 |
| Mirasil ADM-211 | 12 | 12 | 12 | 12 | 12 | 12 |
| Shin Etsu KF8010 | 0 | 1 | 0.5 | 0.3 | 0.2 | 0.1 |
| IDD | q.s | q.s | q.s | q.s | q.s | q.s |
| Total | 100 | 100 | 100 | 100 | 100 | 100 |
| Notes: | ⊕ | X | X | [symbol] | ⊕ | ⊕ |

Gels were formed from all samples except for F and G which contained 0.5% and 1% of the amine end-capped dimethicone polymer (Shin Etsu KF8010). Up to 0.2% by weight of the Shin Etsu KF8010 did not result in loss of the gel. At 0.2% by weight the gel became somewhat fluid, but was still stable. Overall, as the percent amino-dual-end amodimethicone was increased, the viscosity of the gels decreased and eventually precluded firm gel formation above about 0.5% by weight, although fluid gels or viscous fluids were obtained. Interestingly, the compositions according to Samples G and F also display the unique shear thickening rheology and are considered part of the invention even though they were fluid gels or thickened fluids rather than firm gels. This example demonstrates that the dual end amodimethicones can be used to fine tune the firmness of the gel, thickness of the fluid, and the pay-off of the composition.

### Example 5

Gels were constructed from the ingredient shown in Table 5. In each case the Isopropyl Titanium Trisisostearate was Ken-React® KR® TTS. The dimethicone component was varied using a variety of functionalized and unfunctionalized siloxanes. Specifically, the polymers tested were dimethicone (unfunctionalized), dimethiconol, MQ resin (Wacker TMS-803), and amodimethicones (Shine Etsu KF-8005, Mirasil ADM-211 and Shin Etsu KF-867S).

TABLE 5

| Ingredient | E | F | G | H | I | J |
|---|---|---|---|---|---|---|
| Ken-React ® KR ® TTS | 5 | 5 | 5 | 5 | 5 | 8 |
| Mirasil ADM-211 | 10 | — | — | — | — | — |
| Shin Etsu KF867S | — | 10 | — | — | — | — |
| Shin Etsu KF8005 | — | — | 10 | — | — | — |
| Dimethiconol | — | — | — | 10 | — | — |
| MQ resin | — | — | — | — | 10 | — |
| Dimethicone | — | — | — | — | — | 10 |
| IDD | q.s | q.s | q.s | q.s | q.s | q.s |
| Total | 100 | 100 | 100 | 100 | 100 | 100 |
| Notes: | ⊕ | ⊕ | X | X | X | X |

As shown in Table 5, gels were only formed from the amodimethicones (Mirasil ADM-211 and Shin Etsu KF-867S) of Samples E and F. Sample G, which also contained an amodimethicone did not gel. It is believed that the Shin Etsu KF8005 amodimethicone had a FGEW of amine content that was too high to gel this system (see Example 6). Sample H, which contained a dimethiconol also did not gel, nor did the MQ resin of Sample I or the unfunctionailzed dimethicone of Sample J. This study demonstrates the criticality amodimethicones and also the benefit of hydroxy end-capped amodimethicones (Mirasil ADM-211 and Shin Etsu KF-8672) in forming gels with Isopropyl Titanium Trisisostearate.

### Example 6

Gels were constructed from the ingredient shown in Table 6. In each case the Isopropyl Titanium Trisisostearate was Ken-React® KR® TTS. The dimethicone component was an amodimethicone of varying FGEW and viscosity. Shin Etsu KF-874 is a hydroxyl-end-capped amodimethicone and the rest are not.

TABLE 6

| Ingredient | FGEW | Viscosity | E | F | G | H | I | J |
|---|---|---|---|---|---|---|---|---|
| Shin Etsu KF-874 | 19 | 650 | 10 | — | — | — | — | — |
| Shin Etsu KF867S | 17 | 1300 | — | 10 | — | — | — | — |
| Shin Etsu KF861 | 16 | 3500 | — | — | 10 | — | — | — |
| Shin Etsu KF8005 | 110 | 1200 | — | — | — | 10 | — | — |
| Shin Etsu KF880 | 18 | 650 | — | — | — | — | 10 | — |
| Shin Etsu KF393 | 3.5 | 70 | — | — | — | — | — | 10 |
| Ken-React ® KR ® | — | — | 5 | 5 | 5 | 5 | 5 | 5 |
| TTS | | | | | | | | |
| IDD | — | — | q.s | q.s | q.s | q.s | q.s | q.s |
| Total | — | — | 100 | 100 | 100 | 100 | 100 | 100 |
| Notes: | — | — | ⊕ | [symbol] | [symbol] | X | X | X |

Table 6 provides the functional group equivalent weight (FGEW) for the amine content of various amodimethicones in milli-equivalents/g. As shown in Table 6, gels were only formed from Samples E-G. Samples E was the only stable gel on standing at 120° F. for two weeks, as indicated by the symbol ⊕. Sample F was not a stable gel. Sample G was a highly elastic (firm) gel that was not suitable (as is) for use in a cosmetic. Samples H-J did not gel. While it may be possible to form gels with amodimethicones that are not end-capped with hydroxyl groups, the resultant gels have been found to lack stability over prolonged periods and will collapse on freeze thaw cycles. The gelled compositions of the invention are preferably stable over at least one (or at least two) freeze-thaw cycles from −4° F. to 110° F. or from −4° F. to 150° F. The gelled compositions of the invention, when made from amodimethicones that are end-capped with hydroxyl groups, may be stable at 110° F. or 150° F. for two weeks. This example demonstrates the importance of hydroxyl-end-capped amodimethicones (as compared to methyl or alkyl end-capped amodimethicones) in achieving suitable freeze-thaw stability of the resultant gel (or viscous fluids).

### Example 7

Gels were constructed from the ingredient shown in Table 7 to assess the effect of introducing other cosmetic components into the system. In this example, the preservative phenoxyethanol, polar, protic molecule, was added in an amount of 0.5% by weight. In each case the Isopropyl Titanium Trisisostearate was Ken-React® KR® TTS was mixed with amodimethicone (Shin Etsu KF-867S) using IDD as the oil.

TABLE 7

| Ingredient | K | L | M | N | O | P |
|---|---|---|---|---|---|---|
| Ken-React ® KR ® TTS | 5 | 5 | 6 | 7 | 8 | 9 |
| Shin Etsu KF867S | 10 | 10 | 10 | 10 | 10 | 10 |
| Phenoxyethanol | 0 | 0.5 | 0.5 | 0.5 | 0.5 | 0.5 |
| IDD | q.s | q.s | q.s | q.s | q.s | q.s |
| Total | 100 | 100 | 100 | 100 | 100 | 100 |
| Notes: | ⊕ | X | [symbol] | ⊕ | ⊕ | ⊕ |

Comparing Samples K and L, it is seen that the inclusion of polar/protic components such as phenoxyethanol (a preservative) at low levels (e.g., less than 1% by weight) completely destroys the gel. The gel is restored by increasing the amount of Isopropyl Titanium Trisisostearate, with a fluid gel (indicated by \textcircled{×}) being formed at 0.6% (Sample M) and satisfactory gels (indicated by ⊕) formed at 7-9% by weight Isopropyl Titanium Trisisostearate (Samples N-O). This Example demonstrates that minor amounts of common ingredients can have a profound impact on the rheology of the system and that small adjustments in the level of Isopropyl Titanium Trisisostearate can restore the gel.

### Example 8

A composition according to the invention for imparting durable shine to the hair is provided in Table 8.

TABLE 8
Durable Hair Treatment

| Ingredient | Weight % |
|---|---|
| Ken-React ® KR ® TTS | 5 |
| Shin Etsu KF867S | 10 |
| Cyclomethicone (D5) | 30 |
| Dicaprylyl Ether | 5 |
| Trimehtylsiloxyphenyl | 5 |
| Dimethicone | |
| Alcohol SD-40 | 25.5 |

The Ken-React® KR® TTS, Shin Etsu KF867S, and cyclomethicone pentamer were mixed together as Part A to make a gel. The other components, including the shine agent Trimethylsiloxyphenyl Dimethicone, are mixed into the gel. The composition is applied to the hair to impart shine. Bosa Nova gloss analysis may be used to evaluate the shine of the hair. The shine is long-lasting, by which is meant up to 24 hours of normal wear with a loss of 10% or less of the shine.

### Example 9

A composition for imparting renewable shine to the lips is provided in Table 9.

TABLE 9
Blooming Shine Lip gloss

| Ingredient | Weight % |
|---|---|
| Ken-React ® KR ® TTS | 3.5 |
| Shin Etsu KF867S | 10 |
| Cyclomethicone (D5) | 30 |
| MQ resin | 10 |
| D&C Red 7 | 5 |
| IDD | 25.5 |

The Ken-React® KR® TTS, Shin Etsu KF867S, and IDD are mixed with the MQ resin (Belsil TMS 803) and pigments to make a gel. In this example the D&C Red 7 is fluorosilane treated and combined with diphenyl dimethicone. The composition is applied to the lips to impart renewable, long-wearing shine. Bosa Nova gloss analysis may be used to evaluate the shine of the lips. The shine is long-lasting, by which is meant up to 2 or 3 hours of normal wear with a loss or 10% of less of the shine. The shine can be renewed by rubbing the lips.

### Example 10

### Gloss Test

A composition according to the present invention was assessed for shine/gloss intensity using the Gloss Test. The Gloss test measures the gloss intensity, or shine, of a cosmetic film using a gloss meter, which provides gloss values in “gloss units.” For the test described in this example, a MICRO-TRI-GLOSS, Model 4520 (available from BYK-Gardner) was used. The inventive composition tested was Sample B from Example 2. In addition to the inventive composition, commercially available “high shine,” “no stick” products were assessed for shine/gloss using the Gloss Test under the same conditions.

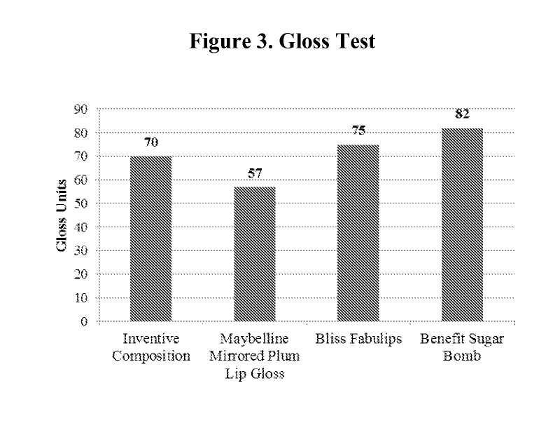

Films of 3 mil thickness of the inventive composition and the three commercially available products were each drawn onto 4 mil thick clear polyester panels. Each of the sample films was flat and free of structures. The films were allowed to dry for one hour prior to measurement. A light source was placed at 20° and the specular reflection was measured. The results are shown in FIG. 3. The data illustrate that a lip gloss according to the invention gave a gloss of about 70 Gloss Units and thus is characterized as high shine (e.g., gloss units greater than about 60). As shown, the gloss is comparable to commercial “high shine” lip glosses, which ranged from 57 to 82 Gloss Units.

### Example 11

### Tack Test

The composition according to the present invention (Sample B of Example 2) and the same commercial “high shine” lip products tested in Example 10 were assessed for tack using the following test. The tack of a cosmetic composition relates to the comfort of the product on the lips (i.e., a lower tack indicates a more comfortable product).

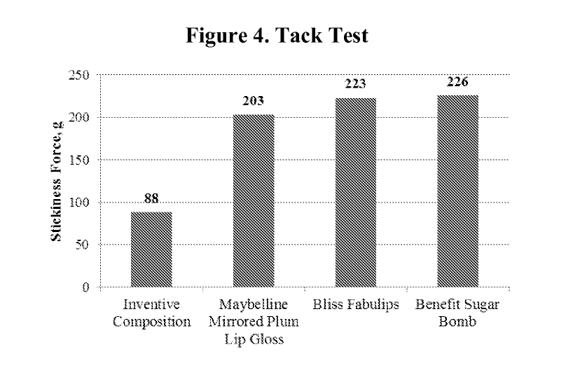

For the tests described in this example, a Texture Analyzer TA-XT2i (available from Texture Technologies, Corp.) was used to measure the force required to separate two pieces of artificial skin coated with a film of the cosmetic. The artificial skin was Vitro-Skin N-19 (available from IMS, Inc.), which was attached to double-sided polyethylene foam tape ( 1/32 of an inch; available from 3M) to mimic the cushioning of human lips, and was cut to the shape of a 1-inch disc. Discs of artificial skin and foam tape were attached to both the probe and the plate base of the texture analyzer. The temperature of the plate base was 34° C., to mimic human lip temperature. Test samples in an amount between 17.78 and 19.76 g/m² were applied evenly to both the artificial skin on the probe and the base plate. The probe was then moved downward towards the base plate until the two pieces of artificial skin were in contact. The probe and the plate base were then pressed together at a force of 100 g for 10 seconds. Following this, the force (g) required to separate fully the two pieces of artificial skin was measured. For each sample, the test was repeated 10 times, and the average force (g) was calculated. The results of these tests are shown in FIG. 4.

As shown in FIG. 4, the inventive composition has substantially lower tack compared to commercial “high shine” lip glosses, even those marketed as no-stick products. The data from Examples 10 and 11 demonstrate that a lip gloss according to the invention maintains a high gloss/shine, while at the same time having a very low tack (e.g., less than about 200 g, or less than about 150 g, or less than about 100 g), thereby providing a more comfortable feel on the lips.

The invention described and claimed herein is not to be limited in scope by the specific embodiments herein disclosed since these embodiments are intended as illustrations of several aspects of the invention. Any equivalent embodiments are intended to be within the scope of this invention. Indeed, various modifications of the invention in addition to those shown and described therein will become apparent to those skilled in the art from the foregoing description. Such modifications are also intended to fall within the scope of the appended claims. All publications cited herein are incorporated by reference in their entirety.

1. A gel or viscous fluid formed from about 3-15% by weight of a titanium alkoxide, about 5-25% by weight of a functionalized amodimethicone polymer selected from hydroxyl-functionalized amodimethicone polymers or alkoxy-functionalized amodimethicone polymers capable of hydrolysis to hydroxyl-functionalized amodimethicone polymers, and about 50-95% by weight of an oil which gels with said titanium alkoxide and hydroxyl-functionalized amodimethicone polymer; wherein a film of said gel or viscous fluid is characterized by a gloss value above about 55 gloss units, and a tack value below about 200 g.

2. The gel or viscous fluid according to claim 1, wherein the titanium alkoxide has the form Ti(OR₁)₃(OR₂) where R₁ is independently selected at each occurrence from C₈₋₃₄ hydrocarbons, and R₂ is selected from C₁₋₇ hydrocarbons.

3. (canceled)

4. (canceled)

5. (canceled)

6. (canceled)

7. (canceled)

8. The gel or viscous fluid according to claim 2, wherein R₁ is isostearyl at each occurrence and R₂ is isopropyl.

9. The gel or viscous fluid according to claim 1, wherein hydroxyl-functionalized amodimethicone polymer comprises end-capped hydroxyl groups.

10. The gel or viscous fluid according to claim 1, wherein the functionalized amodimethicone polymer is characterized by a functional group equivalent weight (FGEW) of amine of about 12-19 milli equivalents per gram.

11. (canceled)

12. The gel or viscous fluid according to claim 1, wherein the functionalized amodimethicone polymer is characterized by a viscosity between about 900 and about 1,300 cps at 25° C.

13. The gel or viscous fluid according to claim 1, wherein the functionalized amodimethicone polymer has the structure: 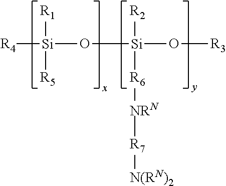
where R₁, R₂, and R₅ are independently selected at each occurrence from C₁₋₈ hydrocarbon radicals, a group L₁, or a group R₈; R₆ and R₇ are independently divalent C₁₋₆ hydrocarbon radicals, R₃ is hydrogen, a group L₁, or a group R₈; and R₄ is —OH, —OR*, or a group L₁ where L₁ is a radical of the form —X^{a}—(CR*₂)ₖ—(CR*═CR*)ₗ—(CR*₂CR*₂O)ₘ—X^{b}—(CR*₂CR*₂O)ₙ—(CR*₂)ₚ—(CR*═CR*)_{q}—OH, where X^{a} and X^{b} are independently either a bond (i.e., absent), —O—, —S—, —NH—, —NR^{N}—; and where “k,” “l,” “m,” “n,” “p,” and “q” are independently at each occurrence integers from 0-4, where R₈ has the structure: 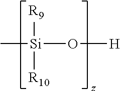
where R₉ and R₁₀ are independently selected from C₁₋₈ hydrocarbon radicals, and where “x,” “y,” and “z” are integers from 0-500, selected such that the polymer has a functional group equivalent weight (FGEW) of amine of about 12-19 milli-equivalents per gram and a viscosity between about 500 and about 5,000 cps at 25° C.,
where R* and R^{N} are independently selected at each occurrence from hydrogen or C₁₋₈ hydrocarbon radicals; with the proviso that said polymer comprises at least two groups —OH.

14. (canceled)

15. (canceled)

16. The gel or viscous fluid according to claim 1, wherein the oil is selected from silicone oils, hydrocarbon oils, fatty alcohols, ester oils, and polyolefins.

17. The gel or viscous fluid according to claim 1, wherein the oil is an optionally hydrogenated polyolefin formed from the polymerization of monomers of the form (R^{a})(R^{b})C═C(R^{c})(R^{d}) where R^{a}, R^{b}, R^{c}, R^{d} are independently selected from hydrogen and C₁₋₆ hydrocarbon radicals.

18. The gel or viscous fluid according to claim 17, wherein the oil is polyisobutylene, hydrogenated polyisobutylene, polybutene, or hydrogenated polybutene.

19. The gel or viscous fluid according to claim 1, further comprising from about 0.05% to about 10% by weight of an amine-functionalized siloxane polymer of the form: 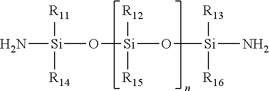
where R₁₁-R₁₆ are independently selected at each occurrence from C₁₋₈ hydrocarbon radicals or a group R₈, where R₈ has the structure: 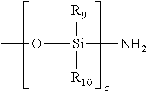
where R₉ and R₁₀ are independently selected from C₁₋₈ hydrocarbon radicals, and where “n” and “z” are integers from 0-300, selected such that the polymer has a viscosity between about 1 and about 2,500 cps at 25° C. and a FGEW between about 100 and about 2,000 g/mol.

20. The gel or viscous fluid according to claim 1, wherein the gel is characterized by a shear thickening (dilatant) rheology at a first range of shear rates and a shear thinning rheology at a second range of shear rates greater than said first range of shear rates.

21. The gel or viscous fluid according to claim 1, wherein the gel is characterized by a shear thickening (dilatant) rheology at a first range of shear rates from about 1 (l/s) to about 15 (l/s) and a shear thinning rheology at a second range of shear rates greater than said first range of shear rates.

22. The gel or viscous fluid according to claim 1, wherein the gel is characterized by a rheotectic rheology at a first shear rate and a thixotropic rheology at a second shear rate greater than said first shear rate.

23. (canceled)

24. A cosmetic composition comprising the gel or viscous fluid according to claim 1, said cosmetic composition further comprising from about 1% to about 35% by pigments.

25. The cosmetic composition according to claim 24, further comprising an ingredient selected from the group consisting of film forming polymers, waxes, co-gellants, thickeners, stabilizers, emulsifiers, humectants, emollients, organic sunscreens, and preservatives.

26. A composition according to claim 24, wherein the composition is a stable gel on heating to 150° F. for two weeks.

27. A composition according to claim 24, wherein the composition is a stable gel on heating to 110° F. for two weeks.

28. A cosmetic composition comprising a gel or viscous fluid formed from about 3-15% by weight of isopropyl triisostearyl titanate, about 5-25% by weight of a hydroxyl-functionalized amodimethicone polymer, about 50-95% by weight of an oil which gels with said isopropyl triisostearyl titanate and hydroxyl-functionalized amodimethicone polymer, and from 0 to about 10% by weight end-capped amine-functionalized siloxane polymer; wherein the hydroxyl-functionalized amodimethicone polymer has the structure: 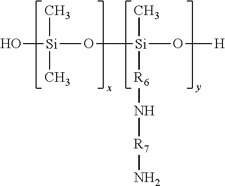
wherein R₆ and R₇ are independently groups-(CH₂)ₖ—, where k is an integer from 1-6; and where “x,” “y” are integers from 1-500, selected such that the polymer has a functional group equivalent weight (FGEW) of amine of about 12-19 milli-equivalents per gram and a viscosity between about 500 and about 2,500 cps at 25° C.;
wherein the end-capped amine-functionalized siloxane polymer of the form: 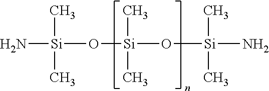
where “n” is an integer from 1-300, selected such that the polymer has a viscosity between about 10 cps and about 2,500 cps at 25° C.; and
wherein a film of said composition is characterized by a gloss value above about 55 gloss units, and a tack value below about 200 g.

29. (canceled)

30. (canceled)

31. (canceled)

32. (canceled)

33. (canceled)

34. (canceled)

35. (canceled)

36. (canceled)

37. (canceled)

38. A method of forming a glossy film on a human integument comprising applying to said integument a deposit of a composition according claim 24, wherein said composition is self-leveling upon the removal of a shear force to regenerate a glossy film.

39. (canceled)

40. (canceled)

41. (canceled)

42. (canceled)
